(19) United States
(12) Patent Application Publication
(10) Pub. No.: US 2013/0121945 A1
(43) Pub. Date: May 16, 2013

(54) THICKENER COMPRISING AT LEAST ONE POLYMER BASED ON ASSOCIATIVE MONOMERS

(71) Applicant: BASF SE, Ludwigshafen (DE)

(72) Inventors: Reinhold J. Leyrer, Dannstadt-Schauernheim (DE); Christofer Arisandy, Ilvesheim (DE); Ouidad Benlahmar, Mannheim (DE)

(73) Assignee: BASF SE, Ludwigshafen (DE)

(21) Appl. No.: 13/670,914

(22) Filed: Nov. 7, 2012

Related U.S. Application Data
(60) Provisional application No. 61/558,440, filed on Nov. 11, 2011.

(51) Int. Cl. C08L 33/10; C11D 3/37; A61Q 19/10; A61Q 5/06; A61Q 5/02; A61Q 19/00; C08L 33/26; A61K 8/81

(52) U.S. Cl. CPC C08L 33/10; C08L 33/26; C11D 3/3757; C11D 3/3765; C11D 3/3773; A61K 8/8164; A61K 8/817; C11D 3/3769; A61Q 5/06; A61Q 5/02; A61Q 19/00; A61Q 19/10
USPC 424/70.16; 524/555; 524/558; 510/475; 510/235; 510/223; 510/119; 510/515; 514/772.4; 510/476; 510/126; 510/237; 510/221; 424/70.17; 514/772.6; 510/158

(57) ABSTRACT

The present invention relates to a thickener comprising

i) at least one polymer preparable by polymerization of
a) at least one water-soluble ethylenically unsaturated monomer comprising at least one anionic monomer and/or at least one nonionic monomer,
b) at least one ethylenically unsaturated associative monomer,
c) optionally at least one crosslinker,
d) optionally at least one chain transfer agent,

ii) at least one activator,

where the ratio of activator to polymer is >10 to 100 [% by weight/% by weight].

### Description

This patent application claims the benefit of pending U.S. provisional patent application Ser. No. 61/558,440 filed on Nov. 11, 2011, incorporated in its entirety herein by reference.

The present invention relates to a thickener comprising at least one polymer and at least one activator where the ratio of activator to polymer is >10:100 [% by weight/% by weight]. The polymer is preparable by polymerizing at least one water-soluble, ethylenically unsaturated monomer comprising at least one anionic monomer and/or at least one nonionic monomer, and at least one ethylenically unsaturated associative monomer. Furthermore, the present invention relates to a process for producing the thickener according to the invention and also surfactant-containing formulations comprising at least one thickener. The invention further provides the use of the surfactant-containing formulation, for example as a softener or as a liquid detergent, and also the use of the thickener, for example as a viscosity modifier.

WO 03/102043 relates to aqueous formulations comprising a cationic polymer prepared from (i) a water-soluble, ethylenically unsaturated monomer or a monomer mixture comprising at least one cationic monomer, (ii) at least one crosslinker in an amount of more than 50 ppm based on component (i), and (iii) at least one chain transfer agent. The aqueous formulations can be used as thickeners in household formulations.

WO 2009/019225 relates to an aqueous dispersion of an alkali-soluble copolymer which is suitable as an associative thickener. The copolymer comprises polymerized-in units of a) at least one ethylenically unsaturated carboxylic acid, b) at least one nonionic ethylenically unsaturated surfactant monomer, c) at least one C₁-C₂-alkyl methacrylate and d) at least one C₂-C₄-alkyl acrylate, where the alkyl chain length averaged over the number of alkyl groups of the alkyl acrylate is 2.1 to 4.0. The associative thickeners can be prepared by emulsion polymerization. The associative thickeners are suitable for use in detergents and cleaners.

Liquid Dispersion Polymer (LDP) compositions are disclosed in WO 2005/097834. These LDP compositions comprise a hydrophilic, water-soluble or swellable polymer with a neutralization content of approximately 25 to approximately 100%, a nonaqueous carrier phase and an oil-in-water surfactant. The hydrophilic, water-soluble or swellable polymer is preferably obtained by polymerization, for example of acrylic add or methacrylic add. The LDP dispersions are suitable for producing microparticulate thickeners, as are used, for example, in aqueous or organic compositions, in particular in personal care or pharmaceutical formulations.

WO 2010/078959 relates to cationic polymer thickeners consisting of a crosslinked water-swellable cationic polymer comprising at least one cationic monomer and optionally nonionic or anionic monomers, where the polymer comprises less than 25% of water-soluble polymer chains, based on the total weight of the polymer. Furthermore, the polymer comprises a crosslinker in a concentration of 500 to 5000 ppm relative to the polymer. The cationic polymer is prepared by inverse emulsion polymerization,

WO 2010/079100 discloses fabric softener compositions comprising polymers according to WO 2010/078959.

US 2008/0312343 relates to inverse latex compositions and to their use as a thickener and/or emulsifier, for example for producing cosmetic or pharmaceutical formulations. The inverse latex compositions comprise at least 50 to 80% by weight of at least one linear, branched or crosslinked organic polymer (p), at least 5 to 10% by weight of an emulsifier system of the water-in-oil type, 5 to 45% by weight of at least one oil and up to 5% water. The polymer P comprises neutral monomers and optionally cationic or anionic monomers. The inverse latex composition can optionally comprise up to 5% by weight of an emulsifier system of the oil-in-water type. The inverse latex compositions can be prepared by inverse emulsion polymerization.

EP-A 172 025 relates to a dispersion in a continuous liquid phase of a polymer which is formed by polymerization of an ethylenically unsaturated monomer comprising a hydrophobic group of at least 8 carbon atoms and an ethylenically unsaturated monomer copolymerizable therewith. The dispersion is stable, essentially anhydrous and comprises at least 40% by weight of polymer. During the polymerization, anionic monomers, for example, can be used as copolymerizable, ethylenically unsaturated monomer. The polymerization can be carried out as an inverse emulsion polymerization.

EP-A 172 724 relates to polymers which are prepared by copolymerization of a) an ethylenically unsaturated monomer comprising a hydrophobic group having at least 8 carbon atoms and b) water-solubly ethylenically unsaturated monomers. All monomers soluble as a mixture in water, and the polymer is prepared by inverse emulsion polymerization. The polymer particles have a dry size of <4 μm. As monomer component b), it is possible to use anionic monomers such as acrylic acid in the form of the free acid or as a water-soluble salt, and also nonionic monomers such as acrylamide.

EP-A 172 723 relates to a process for flocculating a suspension using a water-soluble, essentially linear polymer with a “single point intrinsic viscosity” of >3, The polymer is a copolymer of two or more ethylenically unsaturated monomers comprising at least 0.5% by weight of a monomer which comprises hydrophobic groups. The polymer can also be a cationic polymer.

The problem underlying the present invention consists in the provision of novel thickeners. The object is achieved by the thickeners according to the invention comprising

i) at least one polymer preparable by polymerization
a) at least one water-soluble ethylenically unsaturated monomer comprising at least one anionic monomer and/or at least one nonionic monomer,
b) at least one ethylenically unsaturated associative monomer,
c) optionally at least one crosslinker,
d) optionally at least one chain transfer agent,

ii) at least one activator,

where the ratio of activator to polymer is >10 to 100 [% by weight/% by weight].

The thickeners according to the invention are characterized in that they have advantageous properties with regard to deposition, shear dilution, stabilization and/or viscosity (thickening). Deposition is understood as meaning the deposition of the active ingredients of, for example, a fabric softener on a fiber during a washing operation. Applied to the present invention, this means that, for example, a thickener according to the invention comprising at least one polymer (active ingredient) is present in a fabric softener and the fabric softener is used during or after the washing operation. The thickeners according to the invention promote this deposition of the active ingredient during or after the washing operation to a considerable extent. Particularly good properties with regard to deposition can be achieved when polymers based on predominantly neutral monomers are used which are based on at least one associative monomer, a nonionic monomer such as acrylamide, and optionally an anionic monomer.

When assessing the shear dilution, it is important that the thickener or the corresponding fabric softener in its basic state is viscous and thick whereas it is thin upon stirring. The improved shear dilution has a positive effect on the life and properties of pumps during the production of the fabric softener, promotes convenient dosage for the consumer and promotes the residue-free, use of the fabric softener, especially in the washing machines which have an automatic dosing device. The thickeners according to the invention improve the stability of the thickener per se and that of the corresponding formulation. The settling or creaming of particles is effectively prevented, irrespective of whether they are within the order of magnitude of nanometers, micrometers or millimeters. A contributory factor here is the advantageous yield point of the thickener according to the invention. Moreover, they have the advantage that any redispersion required and the thickening are achieved very quickly.

Thickeners according to the invention in which a mixture of at least two activators is present, where at least one activator has a high HLB value and at least one activator has a low HLB value, are associated with an additional advantage. The combination of such an activator mixture with polymers comprising at least one ethylenically unsaturated associative monomer building block leads to spontaneous phase inversions (within seconds) upon diluting a thickener with water, without requiring an input of additional energy, for example in the form of stirring.

Furthermore, in the case of thickeners according to the invention, it is advantageous that the ratio of associative monomer to the total polymer is relatively low. When using the thickener in surfactant-containing formulations, the effect of the associative monomers is optimal even in amounts of approximately 0.5% by weight (based on the polymer).

If the thickeners according to the invention are prepared by inverse emulsion polymerization in which the temperature is kept constant at least 40° C., a good uniformity of distribution of the associative monomer building blocks within the polymer can be observed. Particularly in the case of small use amounts of, for example, 0.1 to 1% by weight of associative monomers, this is advantageous with regard to the overall aforementioned rheological properties such as thickening, shear dilution, stabilization, and also washing and rinsing effects.

Embodiments of the present invention in which the polymers present in the thickener are prepared using little or no crosslinker are likewise associated with advantages. On account of the relatively high (water-) soluble components of the polymer, resoiling during a washing operation is reduced. Consequently, the article to be washed, even after repeated washing processes, has clean fibers which have been effectively freed from dirt particles, meaning that no graying is detected. No or only very slight adhesion and/or redistribution of dirt particles/polymers on the washed articles is observed.

Within the context of the present invention, the definitions such as C₁-C₃₀-alkyl, as defined, for example, below for the radical R₉ in formula (I), mean that this substituent (radical) is an alkyl radical having a carbon atom number from 1 to 30. The alkyl radical can be either linear or branched and also optionally cyclic. Alkyl radicals which have both a cyclic and a linear component likewise fall within this definition. The same also applies to other alkyl radicals, such as, for example, a C₁-C₄-alkyl radical or a C₁₆⁻C₂₂-alkyl radical. The alkyl radicals can optionally also be mono- or polysubstituted with functional groups such as amino, quaternary ammonium, hydroxy, halogen, aryl or heteroaryl. Unless stated otherwise, the alkyl radicals preferably do not have any functional groups as substituents. Examples of alkyl radicals are methyl, ethyl, n-propyl, isopropyl, n-butyl, isobutyl, 2-ethylhexyl, tertiary-butyl (tert-Bu/t-Bu), cyclohexyl, octyl, stearyl or behenyl.

The present invention is described in more precise terms below,

The thickener according to the invention comprises, as component i), at least one polymer. The polymer is preparable by polymerization of the following components a) and b) and also optionally c) and d).

As component a), at least one water-soluble, ethylenically unsaturated monomer, comprising at least one anionic monomer and/or at least one nonionic monomer is used. Anionic and nonionic monomers per se are known to the person skilled in the art.

If at least one anionic monomer is present in component a), it is preferably selected from acrylic acid, methacrylic acid, itaconic acid, maleic acid or a salt thereof, in particular the anionic monomer is Na acrylate.

If at least one nonionic monomer is present in component a), apart from the nitrogen-containing monomers described below, such as, for example, the compounds according to formula (I), esters of the anionic monomers described above are also suitable as nonionic monomers. Such nonionic monomers are preferably the methyl or ethyl esters of acrylic acid or methacrylic acid such as ethyl acrylate or methyl acrylate. Preference is also given to the corresponding dimethylamino-substituted esters such as dimethylaminoethyl (meth)acrylate,

Preferably, the nonionic monomer is selected from N-vinylpyrrolidone. N-vinylimidazole or a compound according to the formula (I)

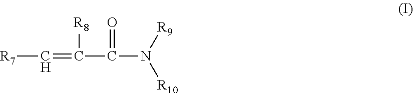

where

R₇ is H C₁-C₄-alkyl,

R₈ is H or methyl, and

R₉ and R₁₀, independently of one are her, are H or C₁-C₃₀-alkyl.

The nonionic monomer is particularly preferably acrylamide, methacrylamide or dialkylaminoacrylamide.

In a preferred embodiment of the present invention, in the polymer, component a) comprises 30 to 99.5% by weight of at least one anionic monomer and 0.5 to 70% by weight of at least one nonionic monomer.

In a further preferred embodiment of the present invention, component a) comprises 100% by weight of at least one nonionic monomer,

In a further preferred embodiment of the present invention, component a) comprises 100% by weight of at least one anionic monomer,

Furthermore, within the context of the present invention, it is preferred that component a) comprises no cationic monomer.

As component b), at least one ethylenically unsaturated associate monomer is used in the polymerization for producing the polymer. Associative monomers per se are known to the person skilled in the art. Suitable associative monomers are described, for example, in WO 2009/019225. Associative monomers are also referred to as surfactant monomers,

Preferably, in the polymer, the ethylenically unsaturated associative monomer according to component b) is selected from a compound according to formula (II)

R—O—(CH₂—CHR′—O)ₙ—CO—CR″=CH₂ (II)

where

R is C₆-C₅₀-alkyl, preferably C₈-C₃₀-alkyl, in particular C₁₆-C₂₂-alkyl,

R′ is H or C₁-C₄-alkyl, preferably H,

R″ is H or methyl,

n is an integer from 0 to 100, preferably 3 to 50, in particular 25,

As component b), particular preference is given to using a compound according to formula (II) in which

R is C₁₆-C₂₂-alkyl,

R′ is H,

R″ is H or methyl and

n is 25,

Compounds according to formula (II) are commercially available in solution, for example under the name Plex 6954 O from Evonik Röhm GmbH. These are methacrylates of fatty alcohol ethoxylates, such as the commercially available Lutensol® AT 25 (BASF SE, Ludwigshafen, Germany).

The radical R in the compounds according to formula (II) can also be present as a mixture of radicals with different chain lengths, such as C₁₆ and C₁₈. One example of this is (3₁₆-C₁₈-fatty alcohol (ethylene glycol)₂₅-ether methacrylate, where both C₁₆ and C₁₈ fatty alcohol radicals (in non-negligible amounts) are present as a mixture, in contrast to this, for example, in the compounds (according to formula (II)) behenyl-25 methacrylate and cetyl-25 methacrylate, the respective radical R is not present as a mixture but as a C₂₂ or C₁₆ chain. Other chain lengths occur only in the form of impurities. The number “25” in these compounds according to formula (II) represents the size of the variables n.

In the preparation of the polymer by polymerization, at least one crosslinker may optionally be present as component c). Suitable crosslinkers are known to the person skilled in the art. Preferably, in the polymer, the crosslinker according to component c) is selected from divinylbenzene; tetraallylammonium chloride; allyl acrylates; allyl methacrylates; diacrylates and dimethacrylates of glycols or polyglycols; butadiene; 1,7-octadiene, allylacrylamides or allylmethacrylamides; bisacrylamidoacetic acid; N,N′-methylenebisacrylamide or polyol polyallyl ethers such as polyallyl sucrose or pentaerythritol triallyl ether. Also suitable as a preferred crosslinker is dialkyldimethylammonium chloride.

Furthermore, during the preparation of the polymer by polymerization, at least one chain transfer agent can he used as component d). Suitable chain transfer agents are known to the person skilled in the art. Preferred chain transfer agents according to component d) are selected from mercaptan, lactic acid, formic acid, isopropanol or hypophosphites.

Suitable polymerization processes for preparing the polymer or the thickener according to the invention comprising at least one polymer, and also any additives or auxiliaries used in the polymerization or in the thickener production process are defined in more detail below.

Preferably, in the thickener according to tho invention, at least one polymer is present which is preparable by polymerization of
a) 20 to 99.99% by weight, preferably 95 to 99.95% by weight (based on the polymer), of at least one water-soluble e.thylenically unsaturated monomer comprising at least one anionic monomer and/or at least one nonionic monomer,
b) 0.01 to 80% by weight, preferably 0.05 to 5% by weight, particularly preferably 0.1 to 1% by weight (based on the polymer) of at least one ethylenically unsaturated associative monomer,
c) 0 to 0.3% by weight, preferably 0.01 to 0.1% by weight (based on the polymer) of optionally at least one crosslinker,
d) 0 to 0.3% by weight, preferably 0.01 to 0.1% by weight (based on the polymer) of optionally at least one chain transfer agent.

In a further embodiment of the present invention, the water-soluble fractions of the polymer are more than 25% by weight (based on the total weight of the polymer), particularly when little or no crosslinker is used in addition to the associative monomer. Preferably, more than 40% by weight, in particular 70 to 100% by weight, of the polymer is soluble in water. The solubility of the polymer is determined by methods known to the person skilled in the art, the polymer present in the thickener according to the invention being admixed with a defined amount of water (see, for example, EP-A 343 840 or preferably the determination method of the sedimentation coefficient in the unit of Svedberg (sved) according to P. Schuck, “Size-distribution analysis of macromolecules by sedimentation velocity ultracentrifugation and Lamm equation modeling, Biophysical Journal 78, (3) (2000), 1606-1619).

Preferably, in this embodiment, the fraction of crosslinker (component c)) used in the polymerization of the polymer is <10% by weight (based on the total amount of components a) to d)). It is particularly preferred not to use any crosslinker in the polymerization of the polymer,

The thickener according to the invention furthermore comprises at least one activator as component ii). Activators per se are known in principle to the person skilled in the art.

Suitable activators are preferably surfactants, for example anionic, nonionic, cationic and/or amphoteric surfactants, which are disclosed, for example, in WO 2009/019225. Preference is given to using anionic and/or nonionic surfactants.

The nonionic surfactants used are preferably fatty alcohol alkoxylates. Fatty alcohol alkoxylates are also referred to as polyalkylene glycol e(hers. Preferred fatty alcohol alkoxylates are alkoxylated, advantageously ethoxylated, in particular primary alcohols having preferably 8 to 18 carbon atoms and on average 1 to 12 mol of ethylene oxide (EO) per mole of alcohol, in which the alcohol radical can be linear or branched, preferably 2-methyl-branched, or can comprise linear and methyl-branched radicals in a mixture, as are usually present in oxo alcohol radicals. Especially preferred, however, are alcohol ethoxylates with linear radicals from alcohols of native or technical origin having 12 to 18 carbon atoms, for example from coconut alcohol, palm alcohol, tallow fatty alcohol or oleyl alcohol—or mixtures, as can he derived, for example, from castor oil—and on average 2 to 8 EO per mole of alcohol. The preferred ethoxylated alcohols include, for example. C₁₂-C₁₄-alcohols with 3 EO, 4 EO or 7 EO, C₉-C₁₁-alcohol with 7 EO, C₁₃-C-₁₅-alcohols with 3 EO, 5 EO, 7 EO or 8 EO, C₁₂-C₁₈-alcohols with 3 EO, 5 EO or 7 EO and mixtures of these, such as mixtures of C₁₂-C₁₄-alcohol with 3 EO and C₁₂-C₁₈-alcohol with 7 EO. The stated degrees of ethoxylation are statistical average values which may be an integer or a fraction for a specific product. Preferred alcohol ethoxylates have a narrowed homolog distribution (narrow range ethoxylates, NRE). In addition to these nonionic surfactants, fatty alcohols with more than 12 EO can also be used. Examples thereof are tallow fatty alcohol with 14 EO, 25 EO, 30 EO or 40 EO. It is also possible to use nonionic surfactants which comprise EO and PO groups together in the molecule. Here, it is possible to use block copolymers with EO-PO block units or PO-EO block units, but also EO-PO-EO copolymers or PO-EO-PO copolymers. It is of course also possible to use mixed alkoxylated nonionic surfactants in which EO and PO units are not blockwise, but in random distribution. Such products are obtainable by the simultaneous action of ethylene oxide and propylene oxide on fatty alcohols.

Moreover, alkyl glycosides or alkyl polyglycosides can also he used as further nonionic surfactants. Alkyl glycosides and alkyl polyglycosides are generally understood by the person skilled in the art as meaning compounds which are composed of at least one alkyl fragment and at least one sugar or polysugar fragment. The alkyl fragments are preferably derived from fatty alcohols with a carbon atom number of 12 to 22, and the sugar fragments are preferably derived from glucose, sucrose or sorbitan. For example, alkyl glycosides of the general formula (1)

R¹O(G)ₓ (1)

can be used, in which R¹ is a primary straight-chain or methyl-branched, in particular 2-methyl-branched, aliphatic radical having 8 to 22, preferably 12 to 18, carbon atoms, and G is a glycoside unit having 5 or 6 carbon atoms, preferably glucose. The degree of oligomerization x, which specifies the distribution of monoglycosides and oligoglycosides, is any number between 1 and 10; preferably, x is 1.2 to 1.4.

A further class of preferably used nonionic surfactants, which are used either as the sole nonionic surfactant or in combination with other nonionic surfactants, are alkoxylated, preferably ethoxylated or ethoxylated and propoxylated, fatty acid alkyl esters, preferably having 1 to 4 carbon atoms in the alkyl chain, in particular fatty acid methyl esters, as are described, for example, in the Japanese patent application JP 58/217598, or which are preferably prepared by the process described in the international patent application WO-A-90/13533.

Nonionic surfactants of the amine oxide type, for example N-cocoalkyl-N,N-dimethylamine oxide and N-tallowalkyl-N,N-dihydroxyethylamine oxide, and of the fatty acid alkanolamide type may also be suitable. The amount of these nonionic surfactants is preferably not more than that of the ethoxylated fatty alcohols, in particular not more than half thereof.

Further suitable surfactants are polyhydroxy fatty acid amides of formula (2),

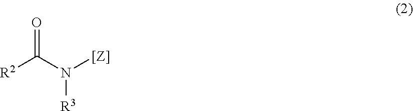

in which R²C(═O) is an aliphatic acyl radical having 6 to 22 carbon atoms, R³ is hydrogen, an alkyl or hydroxyalkyl radical having 1 to 4 carbon atoms and [Z] is a linear or branched polyhydroxyalkyl radical having 3 to 10 carbon atoms and 3 to 10 hydroxyl groups. The polyhydroxy fatty acid amides are known substances which can usually be obtained by reductive amination of a reducing sugar with ammonia, an alkylamine or an alkanolamine, and subsequent acylation with a fatty acid, a fatty acid alkyl ester or a fatty acid chloride.

The group of polyhydroxy fatty acid amides also includes compounds of formula (3)

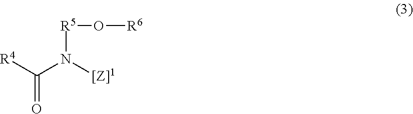

in which R⁴ is a linear or branched alkyl or alkenyl radical having 7 to 12 carbon atoms, R⁵ is a linear, branched or cyclic alkylene radical having 2 to 8 carbon atoms or an arylene radical having 6 to 8 carbon atoms, and R⁶ is a linear, branched or cyclic alkyl radical or an aryl radical or an oxyalkyl radical having 1 to 8 carbon atoms, where C₁-C₄-alkyl or phenyl radicals are preferred, and [Z]¹ is a linear polyhydroxyalkyl radical whose alkyl chain is substituted with at least two hydroxyl groups, or alkoxylated, preferably ethoxylated or propoxylated derivatives of this radical. [Z]¹ is preferably obtained by reductive amination of a sugar, for example glucose, fructose, maltose, lactose, galactose, mannose or xylose. The N-alkoxy- or N-aryloxy-substituted compounds can then be converted to the desired polyhydroxy fatty acid amides, for example, according to WO-A-95107331 by reaction with fatty acid methyl esters in the presence of an alkoxide as catalyst.

The anionic surfactants used are, for example, those of the sulfonate and sulfate type. Suitable surfactants of the sultanate type here are alkylbenzenesulfonates, preferably C₉-C₁₃-alkylbenzenesulfonates, olefinsulfonates, i.e. mixtures of alkene- and hydroxyalkanesulfonates, and also disulfonates, as are obtained, for example, from C₁₂-C₁₈-monoolefins with terminal or internal double bond by sulfonation with gaseous sulfur trioxide and subsequent alkaline or acidic hydrolysis of the sulfonation products.

Also of suitability are alkanesulfonates, preferably secondary alkanesulfonates, which are obtained, for example, from C₁₂-C₁₈-alkanes by sulfochlorination or sulfoxidation with subsequent hydrolysis or neutralization. The esters of α-sulfo fatty acids (ester sulfonates), for example the α-sulfonated methyl esters of hydrogenated coconut fatty acids, palm kernel fatty acids or tallow fatty acids, are also likewise suitable.

Further suitable anionic surfactants are sulfated fatty acid glycerol esters. Fatty acid glycerol esters are to be understood as meaning the mono-, di- and triesters, and mixtures thereof, as are obtained during the preparation by esterification of a monoglycerol with 1 to 3 mol of fatty acid or in the transesterification of triglycerides with 0.3 to 2 mol of glycerol. Preferred sulfated fatty acid glycerol esters here are the sulfation products of saturated fatty acids having 6 to 22 carbon atoms, for example of caproic acid, caprylic acid, capric acid, myristic acid, auric acid, palmitic acid, stearic acid or behenic acid.

Further suitable anionic surfactants are fatty alcohol sulfates, for example alkyl sulfates. Preferred alk(en)yl sulfates are the alkali metal salts, and in particular the sodium salts, of the sulfuric acid monoesters of the C₁₇-C₁₈-fatty alcohols, for example of coconut fatty alcohol, tallow fatty alcohol, lauryl alcohol, myristyl alcohol, cetyl alcohol or stearyl alcohol, or of the C₁₀-C-₂₀-oxo alcohols and those monoesters of secondary alcohols of these chain lengths. Further preferred are alk(en)yl sulfates of said chain lengths which comprise a synthetic straight-chain alkyl radical produced on a petrochemical basis, which have analogous degradation behavior to the equivalent compounds based on fatty-chemical raw materials. In the interests of washing technology, the C₁₂-C₁₆-alkyl sulfates and C₁₂-C₁₅-alkyl sulfates, and also C₁₄-C₁₅-alkyl sulfates are preferred. Also 2,3-alkyl sulfates, which are prepared, for example, according to the US patent specifications U.S. Pat. No. 3,234,258 or 5,075,041 and can be obtained as commercial products from Shell Oil Company under the name DAN^{@}, are suitable anionic surfactants.

The sulfuric acid monoesters of the straight-chain or branched C₇-C₂₁-alcohols ethoxylated with 1 to 6 mol of ethylene oxide, such as 2-methyl-branched C₉-C₁₁-alcohols having on average 3.5 mol of ethylene oxide (EO) C₁₂-C₁₈-fatty alcohols with 1 to 4 EO, are also suitable.

Further suitable anionic surfactants are also the salts of alkylsulfasuccinic acid, which are also referred to as sulfosuccinates or as sulfosuccinic acid esters and which are monoesters and/or diesters of sulfosuccinic acid with alcohols, preferably fatty alcohols and in particular ethoxylated fatty alcohols. Preferred sulfosuccinates comprise C₈-C₁₈-fatty alcohol radicals or mixtures of these. Particularly preferred sulfosuccinates comprise a fatty alcohol radical which is derived from ethoxylated fatty alcohols. In this connection, particular preference is given in turn to sulfosuccinates whose fatty alcohol radicals are derived from ethoxylated fatty alcohols with a narrower homolog distribution. It is likewise also possible to use alk(en)ylsuccinic acid having preferably 8 to 18 carbon atoms in the alk(en)yl chain or salts thereof.

Further suitable anionic surfactants are alkyl carboxylates, for example the sodium salts of saturated or unsaturated fatty acids, where the alkyl radical of the alkyl carboxylate is preferably linear. Within the context of the present invention, the activator is preferably selected from fatty alcohol alkoxylates, alkyl glycosides, alkyl carboxylates, alkylbenzenesulfonates, secondary alkanesulfonates and fatty alcohol sulfates, particularly preferably selected from fatty alcohol alkoxylates. One example of a preferred fatty alcohol alkoxylate is C₆-C₁₇(secondary)-poly(3-6)ethoxylate,

Furthermore, it is preferred within the context of the present invention to use an activator which has a (relatively) high HLB value (Hydrophilic-Lipophilic Balance value). Preferably, the activator has an HLB of 7 to 18, more preferably of 8 to 15 and particularly preferably of 9 to 13.

Activators with a high HLB value are preferably i) fatty alcohol alkoxylates formed from secondary alcohols or mixtures of alcohols having 12 to 18 carbon atoms and ethylene oxide or propylene oxide, and ii) alkyl glycosides formed from sucrose and C₈ to C22-fatty alcohols. Examples of such activators are the commercially available Synperonic 87K from Croda GmbH Herrenpfad-Süd 33, 41334 Nettetal, Germany; Croduret 40 or other ethoxylated hydrogenated castor oils (ricinus oils) such as Etocas 40 or Crodesta F110, all from Croda.

In a further embodiment of the present invention, it is preferred to use a mixture of at least two activators, where at least one activator has a high HLB value and at least one activator has a low HLB value. The activator with a high HLB value preferably has an HLB value of >12 to 20 and the activator with a low HLB value preferably has an HLB value of 1 to 12. In this embodiment, the activator with a high HLB value and the activator with a low HLB value can be present relative to one another in any desired ratios known to a person skilled in the art. Preferably, in the mixture, 20 to 50% by weight of activator with a high HLB value and 50 to 80% by weight of activator with a low HLB value are used. Further preferably, this ratio of activator with a high HLB value to activator with a low HLB value is adjusted such that the overall HLB value is 7 to 18, more preferably 8 to 15 and particularly preferably from 9 to 13.

In these mixtures of at least two activators, the activators with a high HLB value used are preferably alkyl glycosides or polyalkyl glycosides or polyalkyl oligoethylene oxide glycoside based on sucrose or sorbitan and C₈ to C₂₂-fatty alcohols such as polyethylene glycol sorbitan monostearate or polyoxyethylene sorbitan monostearate. Examples of such activators are the commercially available Crillet 1, Crillet 3 or Crodesta F160, all available from Croda. As activators with a low HLB value, preference is given to using alkyl glycosides formed from sucrose or sorbitan and C₈ to C₂₂-fatty alcohols or fatty acids, such as sorbitan laurate or sorbitan stearate. Examples of such activators are the commercially available Crill 1, Crill 3 or Crodesta F10 from Croda.

According to the invention, the ratio of activator to polymer is >10:100 [% by weight/% by weight], preferably 10.5 to 50:100 [% by weight/% by weight], particularly preferably 11.5 to 20:100 [% by weight/% by weight].

In the thickeners according to the invention, further components may also be present in addition to the polymer and the activator. Suitable further components are defined in more detail in the text below within the context of the preparation of the thickener and of the polymer. Suitable further components may be, for example, oils and solvents.

In the thickener according to the invention, the polymer can be present in the oil phase in dispersed form, preferably as an inverse dispersion, water-in-oil dispersion or as a dispersed anhydrous polymer in oil.

The present invention further provides a process for preparing the thickeners according to the invention. Thickener preparation processes per se and processes for preparing a polymer are known to the person skilled in the art. Preferably, the polymer is obtained by an emulsion polymerization, in particular by an inverse emulsion polymerization. Preferably, the polymer is firstly prepared and after the polymerization, preferably by means of inverse emulsion polymerization, the activator is added to give the thickener.

The polymer can be prepared in various ways, preferably by emulsion polymerization, in particular by inverse emulsion polymerization. Inverse emulsion polymerization is understood by the person skilled in the art generally as meaning polymerization processes according to the following definition: the hydrophilic monomers are dispersed in a hydrophobic oil phase. The polymerization takes place directly in these hydrophilic monomer particles by addition of initiator.

Furthermore, it is preferred that, after the inverse emulsion polymerization and before the activator is added, at least some water and at least some of the low-boiling constituents are distilled off from the oil phase, in particular by means of LDP (liquid dispersion polymer) technology. LDP technology per se is known to the person skilled in the art; it is described for example in WO 2005/097834.

Unless stated otherwise, the details below apply to all types of the emulsion polymerization, such as, for example, to the emulsion polymerization in water, which then also constitutes the continuous phase, and in particular also to the inverse emulsion polymerization, in which the hydrophobic oil phase constitutes the continuous phase. A suitable polymerization initiator is used for the polymerization. Redox initiators and/or thermally activatable free-radical polymerization initiators are preferred.

Suitable thermally activatable free-radical initiators or the oxidative component of the redox initiator pair are primarily those of the peroxy and azo type. These include, inter alia, hydrogen peroxide, peracetic acid, t-butyl hydroperoxide, di-t-butyl peroxide, dibenzoyl peroxide, benzoyl hydroperoxide, 2,4-dichlorobenzayl peroxide, 2,5-dimethyl-2,5-bis(hydroperoxy)hexane, perbenzoic acid, t-butyl peroxypivalate, t-butyl peracetate, dilauroyl peroxide, dicapryloyl peroxide, distearoyl peroxide, dibenzoyl peroxide, diisopropyl peroxydicarbonate, didecyl peroxydicarbonate, dieicosyl peroxydicarbonate, di-t-butyl perbenzoate, azobisisobutyronitrile, 2,2′-azobis-2,4-dimethylvaleronitrile, ammonium persulfate, potassium persulfate, sodium persulfate and sodium perphosphate.

The persulfates (peroxodisulfates), in particular sodium persulfate, are most preferred.

When carrying out the emulsion polymerization, the initiator is used in a sufficient amount to initiate the polymerization reaction. The initiator is usually used in an amount of about 0.01 to 3% by weight, based on the total weight of the monomers used. The amount of initiator is preferably about 0.05 to 2% by weight and in particular 0.1 to 1% by weight, based on the total weight of the monomers used.

The emulsion polymerization usually takes place at 35 to 100°C., it can be carried out either as a batch process or in the form of a feed process. In the feed procedure, at least some of the polymerization initiator and optionally some of the monomers are introduced as initial charge and heated to the polymerization temperature, and the remainder of the polymerization mixture is subsequently introduced, usually via a plurality of separate feeds, one or more of which comprise the monomers in pure or emulsified form, continuously or stepwise while maintaining the polymerization. Preferably, the monomer feed takes place in the form of a monomer emulsion. In parallel to the monomer feed, further polymerization initiator can be metered in.

In preferred embodiments, the total amount of initiator is introduced as initial charge, i.e. no further metering of initiator parallel to the monomer feed takes place.

In a preferred embodiment, the thermally activatable free-radical polymerization initiator is therefore introduced in its entirety as initial charge, and the monomer mixture, preferably in the form of a monomer emulsion, is run in. Before the monomer mixture feed is started, the initial charge is brought to the activation temperature of the thermally activatable, free-radical polymerization initiator or a higher temperature. The activation temperature is considered to be the temperature at which at least half of the initiator has decomposed after one hour.

According to another preferred preparation method, the polymer is obtained by polymerization of a monomer mixture in the presence of a redox initiator system. A redox initiator system comprises at least one oxidizing agent component and at least one reducing agent component, in which case heavy metal ions are preferably additionally present in the reaction medium as catalyst, for example cerium salts, manganese salts or iron(II) salts.

Suitable oxidizing agent components are, for example, peroxides and/or hydroperoxides such as hydrogen peroxide, tert-butyl hydroperoxide, cumene hydroperoxide, pinane hydroperoxide, diisopropylphenyl hydroperoxide, dicyclohexyl percarbonate, dibenzoyl peroxide, dilauroyl peroxide and diacetyl peroxide. Hydrogen peroxide and tert-butyl hydroperoxide are preferred.

Suitable reducing agent components are alkali metal sulfites, alkali metal dithionites, alkali metal hyposulfites, sodium hydrogensulfite, Rongalit C (sodium formaldehyde sulfoxylate), mono- and dihydroxyacetone, sugars (e.g. glucose or dextrose), ascorbic acid and its salts, acetone bisulfite adduct and/or an alkali metal salt of hydroxymethanesulfinic acid, sodium hydrogensulfite or sodium metabisulfite are preferred.

Also suitable as reducing agent component or catalyst are iron(II) salts such as e.g. iron(II) sulfate, tin(II) salts such as e.g. tin(II) chloride, titanium(III) salts such as titanium(III) sulfate.

The use amounts of oxidizing agent are 0.001 to 5.0% by weight, preferably from 0.005 to 1.0% by weight and particularly preferably from 0.01 to 0.5% by weight, based on the total weight of the monomers used, Reducing agents are used in amounts of from 0.001 to 2.0% by weight, preferably from 0.005 to 1.0% by weight and particularly preferably from 0.01 to 0.5% by weight, based on the total weight of the monomers used.

A particularly preferred redox initiator system is the sodium peroxodisulfate/sodium hydrogensulfite system, e.g. 0.001 to 5.0% by weight of sodium peroxodisulfate and 0.001 to 2.0% by weight of sodium hydrogensulfite, in particular 0.005 to 1.0% by weight of sodium peroxodisulfate, and 0.005 to 1.0% by weight of sodium hydragensulfite, particular preferably 0.01 to 0.5% by weight of sodium peraxoclisulfate and 0.01 to 0.5% by weight of sodium hydrogensulfite.

A further particularly preferred redox initiator system is the t-butyl hydroperoxide/hydrogen peroxide/ascorbic acid system, e.g. 0.001 to 5.0% by weight of t-butyl hydroperoxide, 0.001 to 5.0% by weight of hydrogen peroxide and 0.001 to 2.0% by weight of ascorbic acid, in particular 0.005 to 1.0% by weight of t-butyl hydroperoxide, 0.005 to 1.0% by weight of hydrogen peroxide and 0.005 to 1.0% by weight of ascorbic acid, particularly preferably 0.01 to 0.5% by weight of t-butyl hydroperoxide, 0.01 to 0.5% by weight of hydrogen peroxide and 0.01 to 0.5% by weight of ascorbic acid.

Preferably, the polymer is prepared by inverse emulsion polymerization by firstly preparing an aqueous phase of the water-soluble components and an oil phase separately from one another. Subsequently, the two phases are mixed with one another to give a water-in-oil dispersion. The mixture is polymerized by adding a redox initiator system; optionally, a thermal initiator can then also be added or, if already present, be thermally activated.

In the aqueous phase, preferably a chain transfer agent, a crosslinker, an anionic monomer and/or a neutral monomer and also optionally the associative monomer are present, and also optionally further components. Suitable further components are, for example, complexing agents for salts such as pentasodium diethylene-triaminepentaacetic acid,

In the oil phase, preferably an emulsifier, a stabilizer, a high-boiling oil, a low-boiling oil and/or optionally the associative monomer are present. Furthermore, a nonionic monomer may optionally be present in the oil phase.

Emulsifiers, stabilizers, low-boiling oils and high-boiling oils as such are known to the person skilled in the art. These compounds can be used individually or in the form of mixtures.

Typical emulsifiers are anionic emulsifiers such as, for example, sodium lauryl sulfate, sodium tridecyl ether sulfates, dioctyl sulfasuccinate sodium salt and sodium salts of alkylaryl polyether sulfonates; and nonionic emulsifiers such as, for example, alkylaryl polyether alcohols and ethylene oxide-propylene oxide copolymers. Sorbitan trioleate is likewise suitable as an emulsifier.

Preferred emulsifiers have the following general formula:

R—O—(CH₂—CHR′—O)ₙ—X

in which R is C₆-C₃₀-alkyl.

R′ is hydrogen or methyl,

X is hydrogen or SO₃M,

M is hydrogen or an alkali metal, and

n is an integer from 2 to 100.

Suitable stabilizers are described, for example, in EP-A 172 025 or EP-A 172 724. Preferred stabilizers are copolymers of stearyl methacrylate and methacrylic acid.

Suitable high-boiling oils are, for example, 2-ethylhexyl stearate and also hydroheated heavy naphtha, and suitable low-boiling oils are, for example, dearomatized aliphatic hydrocarbons or mineral oils of low viscosity.

In a preferred embodiment of the present invention, during the inverse emulsion polymerization, component b) (at least one ethylenically unsaturated associative monomer) is added to the oil phase.

During the inverse emulsion polymerization, the temperature can be kept constant or it can also increase. The increase in temperature can be carried out continuously or in stages. Thus, for example, the temperature during the polymerization can increase by 0.2 to 10° C. per minute, preferably from 1 to 3° C. per minute. The temperature increase is controlled by the rate at which the initiator is added. The temperature starting value can be 0 to 30° C., preferably 10 to 20° C.

In another embodiment of the present invention, the temperature during the inverse emulsion polymerization is kept constant (cold procedure), the temperature is 0 to 30° C., preferably 10 to 20°C. In a further embodiment of the present invention, the temperature is kept constant in a higher temperature range (warm procedure). The temperature during the polymerization is 40 to 150° C., preferably 70 to 120° C.

In a particularly preferred embodiment of the present invention, the temperature is kept constant during the inverse emulsion polymerization, the temperature being at least 40° C., preferably 50 to 90° C.

If, within the context of the present invention, the temperature is kept constant during a polymerization, in particular during an inverse emulsion polymerization, this means that the temperature is kept at a constant value from the start of the polymerization. Fluctuations of +/−5° C., preferably +/−2° C. and in particular +/−1° C. during the polymerization process are considered to be a constant temperature (based on the desired constant temperature value). The temperature is kept constant until the polymerization is complete; preferably, this is the case after a conversion of more than 90% of the monomers used, more preferably more than 95% by weight and particularly preferably at complete conversion (100% by weight). The temperature can be kept constant by dissipating the heat of reaction which arises by cooling. The start of the polymerization is normally the addition of the polymerization initiator, preferably the addition of a redox initiator system. Normally, the system is firstly heated to the desired temperature and a constant temperature is awaited while stirring. Then, the polymerization initiator is added, as a result of which the polymerization process is set in motion. In one embodiment of the present invention, the temperature is kept constant at a value above the melting point of the associative monomer used.

The present invention further provides surfactant-containing acidic formulations comprising at least one thickener according to the invention as per the definitions above. The pH of the formulation is 1 to <7.

The present invention further provides surfactant-containing alkaline formulations comprising at least one thickener according to the invention as per the definitions above. The pH of the formulation is 7 to 13.

The surfactant-containing acidic or alkaline formulations according to the invention can comprise further ingredients known to the person skilled in the art. Suitable ingredients comprise one or more substances from the group of builders, bleachers, bleach activators, enzymes, electrolytes, nonaqueous solvents, pH modifiers, fragrances, perfume carriers, fluorescent agents, dyes, hydrotropes, foam inhibitors, silicone oils, antiredeposition agents, optical brighteners, graying inhibitors, antishrink agents, anticrease agents, dye transfer inhibitors, antimicrobial active ingredients, germicides, fungicides, antioxidants, corrosion inhibitors, antistats, ironing aids, phobicization and impregnation agents, swelling and antislip agents, and UV absorbers.

The present invention further provides the use of a surfactant-containing acidic formulation according to the invention in hair cosmetics, in hairstyling, as a shampoo, as a softener, as a care composition, as a conditioner, as a skin cream, as a shower gel, as a fabric softener for laundry, or as an acidic cleaner, preferably for the toilet or the bath.

The present invention further provides the use of a surfactant-containing alkaline formulation as a care composition, as a liquid detergent or as a dishwashing detergent for machine washing or hand washing,

The present invention further provides the use of the thickener according to the invention as a viscosity modifier, for optimizing shear dilution, as a thickening agent, for stabilizing suspended ingredients with a size in the range from nanometers to millimeters and/or in surfactant-containing acidic or alkaline formulations.

In the description including the examples, the following abbreviations are used:
Monomers
   ACM Acrylamide
   AA Acrylic acid
   MAA Methacrylic acid
   NaAc Sodium acrylate
   BEM Behenyl-25 methacrylate
   MBA Methylenebisacrylamide (crosslinker)
   TAAC Tetraallylammonium chloride (crosslinker)
   NaHP Sodium hypophosphite (chain transfer agent)
   C16EO25MAc C₁₆-C₁₈-Fatty alcohol-(ethylene glycol)₂₅, ether methacrylate
Others
   pphm Parts per hundred parts of monomers (based on components a) and b))
   demin. Demineralized

The invention is illustrated below by reference to the examples,

### EXAMPLES

### Comparative example C1

Synthesis of a thickener/polymer starting from anionic monomers without associative monomer, but with crosslinker and chain transfer agent and also increasing polymerization temperature.

An aqueous phase of water-soluble components is prepared by mixing the following components:

250.24 g (139.02 pphm) of water,

0.89 g (0.49 pphm) of pentasodium diethylenetriaminepentaacetic acid,

11.05 g (0.06 pphm) of methylenebisacrylamide (1% in water),

180 g (100 pphm) of acrylic acid and

146.8 g (40.78 pphm) of NaOH (50% in water)

Use NaOH (50% in water) to adjust the water phase to pH 5.5,

An oil phase is prepared by mixing the following components:

20.62 g (8.59 pphm) of sorbitan monooleate (75% in hydroheated heavy naphtha (petroleum) [Isopar G])

93.19 g (12.27 pphm) of a polymeric stabilizer: stearyl methacrylate-methacrylic acid copolymer (23.7% in hydroheated heavy naphtha [lsopar G]),

120.24 g (66.8 pphm) of mineral oil of low viscosity (Kristol M14) and

236.28 g (131.27 pphm) of hydroheated heavy naphtha [isopar G].

The two phases are mixed in a ratio of 55.6 parts of aqueous phase to 44.4 parts of oil phase with high shear to produce a water-in-oil emulsion. The resulting water-in-oil emulsion is introduced into a reactor equipped with nitrogen spray line, stirrer and thermometer. The emulsion is purged with nitrogen, as a result of which the oxygen is removed, and is cooled to 20° C.

The polymerization is achieved by adding a redox pair composed of

13 g (0.014 pphm) of sodium metabisulfite, (0.2% in hydroheated heavy naphtha (petroleum) [Isopar G] and

13 g (0.014 pphm) of tertiary-butyl hydroperoxide (0.2% in hydroheatedheavy naphtha (petroleum) [Isopar G].

The redox pair is added stepwise such that a temperature increase of 2° C./min takes place. Once the isotherm has been reached, a free radical initiator (2,2′-azobis(2-methylbutyronitrile), CAS: 13472-08J) is added in 2 steps (the 2nd step after 45 min) and the emulsion is kept at 85° C. for 75 minutes.

Vacuum distillation is used to remove water and low-boiling constituents of the oil phase (Isopar G).

Mineral oil of low viscosity (Kristol M14) is added to this product in order to achieve a solids content of 54%. To this product 8% (based on the total mass fraction of this product) of a fat-containing alcohol alkoxylate (C12/15 alcohol alkoxylate [Synperonic 87K™]) is added to produce a thickener (dispersion) with 50% polymer solids fraction. The ratio of activator to polymer is thus 16.0:100 [% by weight/% by weight].

### Example 1

Thickeners/polymers starting from anionic monomers with associative monomer and constant polymerization temperature:

### Example 1.1

An aqueous phase of water-soluble components is prepared by mixing the following components:

246.13 g (140.65 pphm) of water,

0.86 g (0.49 pphm) of pentasodium diethylenetriaminepentaacetic acid,

174.13 g (99.5 pphm) of acrylic acid and

154.26 g (44.07 pphm) NaOH (50% in water)

Use NaOH (50% in water) to adjust the water phase to pH 5,5.

An oil phase is prepared by mixing the following components:

20.05 g (8.59 pphm) of sorbitan monooleate (75% in hydroheated heavy naphtha (petroleum) [Isopar G])

90.6 g (12.27 pphm) of polymeric stabilizer: stearyl methacrylate-methacrylic acid copolymer (23.7% in hydroheated heavy naphtha [lsopar G]),

119.03 g (68.02 pphm) of mineral oil of low viscosity (Kristol M14) and

229.72 g (131,27 pphm) of hydroheated heavy naphtha [Isopar G]

1.09 g (0.5 pphm) of associative monomer: 60% by weight of C16EO25Mac: comprised in the commercial product Plex 6954-O (with 20% by weight of methacrylic acid, 20% by weight of water).

The two phases are mixed in a ratio of 55.6 parts of aqueous phase to 44.4 parts of oil phase with high shear to produce a water-in-oil emulsion. The resulting water-in-oil emulsion is introduced into a reactor equipped with nitrogen spray line, stirrer and thermometer. The emulsion is purged with nitrogen while heating to 50° C., as a result of which the oxygen is removed.

The polymerization is achieved y adding a redox pair composed of

13.6 g (0.016 pphm) of sodium metabisulfite (0.2% in water) and

13.6 g (0.016 pphm) of tertiary-butyl hydroperoxide (0.2% in water).

The redox pair is added at 50° C. over the course of 2 hours. After this, the mixture is heated to 85° C. and then, in 2 steps, (the 2nd step after 45 min) a free radical initiator (2,2′-azobis(2-methylbutyronitrile), CAS: 13472-08-7) is added and the emulsion is kept at 85° C. for 75 minutes.

Vacuum distillation is used to remove water and low-boiling constituents of the oil phase (Isopar G),

Mineral oil of low viscosity (Kristol M14) is added to this product in order to achieve a solids content of 54%. To this product 8% (based on the total mass fraction of this product) of a fat-containing alcohol alkoxylate (C12/15 alcohol alkoxylate. [Synperonic 87K™]) is added to produce a thickener (dispersion) with 50% polymer solids fraction. The ratio of activator to polymer is thus 16.0:100 [% by weight/% by weight].

The examples below as per table 1 are prepared as in example 1.1 taking into consideration the stated changes in the monomer composition. The associative monomer C16EO25MAc is added to the oil phase. The commercial product Plex 6954 O is used; this comprises 60% by weight of associative monomer and, as solvent, water and MAA in the ratio of ca. 1:1. The weight data in table 1 refers to the amount of associative monomer without solvent. The ratio of activator to polymer in all examples as per table 1 is in each case 16.0:100 [% by weight/% by weight]; unless stated otherwise, the respective thickeners (dispersion) have 50% polymer solids fraction.

TABLE 1

| | C16EO25MAc | Na | | MBA | |
|---|---|---|---|---|---|
| Examples | (pphm) | acrylate | Acrylamide | (pphm) | Remarks |
| 1.1 | 0.38 | 99.5 | | — | |
| 1.2 | 0.38 | 99.5 | | 0.06 | |
| 1.3 (comp.) | — | | 100 | — | |
| 1.4 | 0.38 | | 99.5 | — | |

### Example 2

Thickeners/polymers starting from anionic monomers with associative monomer and also increasing polymerization temperature:

The following examples as per table 2 are prepared as in comparative example C1 taking into consideration the stated changes in the monomer composition. The associative monomer C16EO25MAc is added to the oil phase. The commercial product Plex 6954 O is used; this comprises 60% by weight of associative monomer and, as solvent, water and MAA in the ratio of ca. 1:1. The weight data in table 2 refers to the amount of associative monomer without solvent. The ratio of activator to polymer in all examples as per table 2 is in each case 16.0:100 [% by weight/% by weight]; unless stated otherwise, the respective thickeners (dispersion) have 50% polymer solids fraction.

TABLE 2

| | C16EO25MAc | Na | Acryl- | MBA | |
|---|---|---|---|---|---|
| Examples | (pphm) | acrylate | amide | (pphm) | Remarks |
| 2.1 | 1.5 | 98 | | 0.06 | |
| 2.2 | 0.38 | 99.5 | | 0.06 | |
| 2.3 | 1.5 | 98 | | 0.06 | Starting |
| | | | | | temperature |
| | | | | | at 14° C. |
| 2.4 | 1.5 | 98 | | — | |
| 2.6 | 0.38 | 99.5 | | — | |
| 2.5 (comp.) | — | 100 | | 0.2 | |
| C1 | — | 100 | | 0.06 | |
| 2.7 (comp.) | — | — | 100 | — | |
| 2.8 | 0.38 | — | 99.5 | — | |

General Measurement Methods:

Unless stated otherwise, the following general measurement methods are used in the examples below:

Determination of Viscosity

Taking into consideration the procedures according to DIN 51550, DIN 53018, DIN 53019, the Brookfield model DV II viscometer is used, unless stated otherwise in the tables below at a speed of 20 revolutions per minute with spindle No. 6, to measure the stated viscosities in mPa·s.

Determination of Shear Dilution

Measurement is carried out in an ASC (automatic sample changer) rotary rheometer from Antonpaar, with the CC27 cylinder geometry, the radius of the measurement body of 13.33 mm and the radius of the measurement cup of 14.46 mm. The measurement temperature is 23° C. The samples are measured at steady-state shear starting at a low shear increasing to high shear (0.01 s⁻¹-1000 s⁻¹)and back again (1000 s⁻¹-0.01 s⁻¹).

### Example 3

Use of the Thickeners/Polymers in Water

The thickeners are slowly added to distilled water as per table 3 at room temperature and stirred until the formulation has homogenized. The aqueous formulations obtained as a result comprise, according to table 3, either 1.0% by weight of polymer to 99.0% by weight of water or 0.5% by weight of polymer to 99.5% by weight of water. The results are summarized in table 3.

TABLE 3
Rheology of thickeners/polymers starting from anionic monomers in water,
measured 5 minutes after preparing the formulation

| | | | Thickener | Brookfield |
|---|---|---|---|---|
| Example | | Thickener | concentration | spindle 6 |
| No. | Formulation | No. | (%) | (20 rpm)/mPas |
| 3.1 (comp.) | Water | V1 | 1.0 | 13250 |
| 3.7 (comp.) | Water | 2.5 (comp.) | 1.0 | 9600 |
| 3.2 | Water | 2.1 | 1.0 | 22000 |
| 3.3 | Water | 2.2 | 1.0 | 14050 |
| 3.4 | Water | 2.3 | 1.0 | 23000 |
| 3.5 | Water | 1.1 | 1.0 | 15600 |
| 3.6 | Water | 1.2 | 1.0 | 18300 |
| 3.9 (comp.) | Water | 2.7 (comp.) | 2.0 | 100 |
| 3.10 | Water | 2.8 | 2.0 | 250 |
| 3.11 (comp.) | Water | 1.3 (comp.) | 2.0 | 200 |
| 3.12 | Water | 1.4 | 2.0 | 450 |

If an increasing amount of associative monomer is incorporated into the polymer, then the thickening performance increases more and more considerably compared with C1 without associative monomer. The lower the starting temperature during the polymerization according to example 2, the greater the thickening performance. The procedure at a constant polymerization temperature of 50° C. produces, for an otherwise identical monomer composition, an increased thickening performance. The last four examples of table 3 relate to acrylamide-containing polymers.

### Example 4.1

Use of the thickeners/polymers in standard formulations of care compositions

The care composition formulation (P1) comprises the ingredients as in table 4.1. P1 is prepared by heating phases A and B in each case to 80° C. The two phases are then combined and homogenized. Then, with stirring, the mixture is cooled to approximately 40° C., whereupon phase C is added and the mixture is homogenized.

TABLE 4.1.1
Ingredients P1

| Phase | Trade name | INCI name | % |
|---|---|---|---|
| A | 1,2 Propylene glycol | Propylene glycol | 5.00 |
| | Care | | |
| | Water dem. | Aqua | ad 100 |
| B | Cremophor A 6 | Ceteareth-6, | 2.00 |
| | | Stearyl Alcohol | |
| | Cremophor A 25 | Ceteareth-25 | 2.00 |
| | Luvitol EHO | Cetearyl | 5.00 |
| | | Ethylhexanoate | |
| | Paraffin oil thick-liq. | Mineral oil | 5.00 |
| | Lanette 16 | Cetyl Alcohol | 2.50 |
| C | Euxyl K 300 | Preservative | 0.50 |

Addition of the Thickener to the Care Composition Formulation P1:

The thickeners according to the invention listed in table 4.1.2 or thickeners according to the comparative examples are slowly added, at room temperature, to the care composition formulation P1 and stirred until the formulation is homogenized. The care composition formulations obtained in this way comprise the stated concentration of thickener in % by weight relative to 100% by weight of the resulting care composition formulations.

The Brookfield viscosity is measured one day after the preparation. The results are summarized in table 4.1.2.

TABLE 4.1.2
Thickener performance and shear dilution in care compositions
Rheology of fabric softeners comprising thickeners/polymers starting
from anionic monomers:

| | | | Thickener | Brookfield |
|---|---|---|---|---|
| Example | | Thickener | concentration | spindel 6 |
| No. | Formulation | No. | (%) | (20 rpm)/mPas |
| 4.1.1 | P1 | V1 | 1.6 | 13600 |
| (comp.) | | | | |
| 4.1.2 | P1 | V1 | 2.4 | 19250 |
| (comp) | | | | |
| 4.1.3 | P1 | V1 | 3.2 | 24300 |
| (comp.) | | | | |
| 4.1.4 | P1 | 2.1 | 1.6 | 17400 |
| 4.1.5 | P1 | 2.1 | 2.4 | 33700 |
| 4.1.6 | P1 | 2.1 | 3.2 | 59400 |

If an increasing amount of associative monomer is incorporated into the polymer, then the thickening performance increases more and more considerably compared with V1 without associative monomer.

### Example 4.2

Use of the Thickeners/Polymers in Standard Formulations of Fabric Softener W3

W3: Preparation of a methyltris(hydroxyethyl)ammonium-di-tallow-fatty acid ester methosulfate, partially hydrogenated fabric softener (5.5% active fraction):

The fabric softener has a pH of 2.7 and comprises 5.5% by weight of methyltris(hydroxyethyl)ammonium-di-tallow-fatty acid ester methosulfate (partially hydrogenated) and 94.5% by weight of deionized water.

Addition of the thickener to the fabric softener formulation W3:

The thickeners (see table 4.2) according to examples 1 and 2 and the comparative examples are slowly added, at room temperature, to the respective fabric softener formulation and stirred until the formulation has homogenized.

The Brookfield viscosity is measured one day after the preparation. The results are summarized in table 4.2.

TABLE 4.2
Thickener performance in fabric softener W3
Rheology of fabric softeners
comprising thickeners/polymers starting from neutral monomers:

| | | | Thickener | Brookfield |
|---|---|---|---|---|
| Example | | Thickener | concentration | spindel 6 |
| No. | Formulation | No. | (%) | (20 rpm)/mPas |
| 4.2.1 | W3 | 1.3 | 1.0 | 400 |
| (comp.) | | (comp.) | | |
| 4.2.2 | W3 | 1.4 | 1.0 | 1900 |
| 4.2.3 | W3 | 2.7 | 1.0 | 600 |
| (comp.) | | (comp.) | | |
| 4.2.4 | W3 | 2.8 | 1.0 | 1100 |

If associative monomer is incorporated into the polymer, then the thickening performance increases more and more considerably compared with the comparative examples without associative monomer.

### Example 5

In table 5 below, the storage stability of the thickeners according to the invention is investigated. It is found that the thickeners according to the invention are considerably more stable.

TABLE 5
Storage stability of thickeners/polymers starting from anionic monomers:

| | Thickener | Immediate | Precipitate after 4 |
|---|---|---|---|
| Example | No. | precipitate | days at 40° C. |
| 5.1 (comp.) | V1 | None | Considerable, not |
| | | | redispersible |
| 5.2 | 2.1 | None | None |

Significant improvement, i.e. reduction in sediment, by thickeners according to the invention.

### Example 6

Storage stability of care composition formulation (P1) according to example 4 comprising thickeners/polymers starting from anionic monomers:

Significant improvement, i.e., reduction in sediment, by thickeners according to the invention. After almost three months, significant sediment evident for formulation with thickener V1 and only minimal sediment evident for formulation with thickener 2.1 (storage at RT).

### Example 7

Thickeners/polymers starting from anionic monomers with associative monomer and influence of the amount of activator on the thickening rate in aqueous formulations:

Examples 7.1 to 7.5 listed in table 6 are prepared in accordance with example 2.1, the amount of activator added after the distillation being varied to correspond to the activator concentration (A %) in the thickener given in table 11 (all data in % by weight based on the amount of anionic polymer in the thickener). All of the thickeners (dispersion) prepared in this way have a polymer solids fraction of 50%. The thickeners are then added to the water with stirring. The aqueous formulations obtained in this way comprise 1% by weight of the thickener to 99% by weight of water, i.e. 0.5% by weight of anionic polymer to 99.5% by weight of water. comp. means comparative example.

TABLE 6

| | | Thickening rate (Brookfield visc. mPas * s) | | | | | | | |
|---|---|---|---|---|---|---|---|---|---|
| | | of the aqueous formulations | | | | | | | |
| Examples | A % | 1 min | 2 min | 3 min | 5 min | 10 min | 20 min | 40 min | 1 h |
| 7.1 | 2.0 | 128 | 240 | 512 | 1640 | 9300 | 20000 | 27500 | 30000 |
| (comp.) | | | | | | | | | |
| 7.2 | 6.0 | 4590 | 6750 | 10300 | 16700 | 23300 | 26200 | 30100 | 35600 |
| (comp.) | | | | | | | | | |
| 7.3 | 16.0 | 17000 | 18400 | 19900 | 22400 | 26900 | 35200 | 40400 | 40400 |
| 7.4 | 20.0 | 32000 | 32800 | 34000 | 36400 | 38400 | 41200 | 42000 | 42000 |
| 7.5 | 34.0 | 41600 | 42000 | 42400 | 42400 | 42400 | 42400 | 42400 | 42400 |

With associative monomer, an amount of activator above 10% is required in order to achieve a rapid thickening performance (above 40% based on the and thickening within 1 minute).+

1.-20. (canceled)

21. A thickener comprising
i) at least one polymer prepayable by polymerization of
a) at least one water-soluble ethylenically unsaturated monomer comprising at least one anionic monomer or at least one nonionic monomer,
b) at least one ethylenically unsaturated associative monomer,
c) optionally at least one crosslinker,
d) optionally at least one chain transfer agent,
ii) at least one activator,
where the ratio of activator to polymer is >10 to 100 [% by weight/% by weight].

22. The thickener according to claim 21, wherein component a) comprises at least one anionic monomer and at least one nonionic monomer.

23. The thickener according to claim 21, wherein, in the polymer, component a) comprises at least one anionic monomer, where the anionic monomer is selected from acrylic acid, methacrylic acid, itaconic acid, maleic acid or a salt thereof.

24. The thickener according to claim 23, wherein the anionic monomer is Na acrylate.

25. The thickener according to claim 21, wherein the water-soluble fractions of the polymer are more than 25% by weight (based on the total weight of the polymer).

26. The thickener according to claim 21, wherein, in the polymer, component a) comprises at least one nonionic monomer, where the nonionic monomer is selected from N-vinylpyrrolidone, N-vinylimidazole or a compound according to the formula (I) 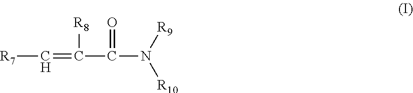
where
R₇ is H or C₁-C₄-alkyl,
R₈ is H or methyl, and
R₉ and R₁₀, independently of one another, are H or C₁-C₃₀-alkyl.

27. The thickener according to claim 21, wherein, in the polymer, the ethylenically unsaturated associative monomer (component b) is selected from a compound according to formula (II)
R—O—(CH₂—CHR′—O)ₙ—CO—CR″═CH₂ (II)
where
R is C₆-C₅₀-alkyl,
R′ is H or C₁-C₄-alkyl,
R″ is H or methyl,
n is an integer from 0 to 100.

28. The thickener according to claim 21, wherein, in the polymer, the crosslinker (component c) is selected from
divinylbenzene; tetraallylammonium chloride; allyl acrylates; allyl methacrylates; diacrylates and dimethacrylates of glycols or polyglycols; butadiene; 1,7-octadiene, allylacrylamides or allylmethacrylamides; bisacrylamidoacetic acid; N,N′-methylenebisacrylamide or polyol polyallyl ethers such as polyallyl sucrose or pentaerythritol triallyl ether.

29. The thickener according to claim 21, wherein, in the polymer, the chain transfer agent (component d) is selected from mercaptans, lactic acid, formic acid, isopropanol or hypophosphites.

30. The thickener according to claim 21, wherein the activator is selected from fatty alcohol alkoxylates, alkyl glycosides, alkyl carboxylates, alkylbenzenesulfonates, secondary alkanesulfonates and fatty alcohol sulfates, preferably selected from fatty alcohol alkoxylates.

31. The thickener according to claim 21, wherein a mixture of at least 2 activators is used, where at least one activator has an HLB value (hydrophilic-lipophilic balance value) of >12 to 20 and at least one activator has an HLB value of from 1 to 12.

32. The thickener according to claim 21, wherein the polymer is present in the oil phase in dispersed form.

33. The thickener according to claim 32, wherein the dispersed form is an inverse dispersion, water-in-oil dispersion, or a dispersed anhydrous polymer in oil.

34. A process for preparing a thickener according to claim 21, wherein the polymer is obtained by an emulsion polymerization.

35. The process according to claim 34, wherein the polymer is obtained by an inverse emulsion polymerization.

36. The process according to claim 35, wherein, after the inverse emulsion polymerization and before the activator is added, at least some water and at least some of the low-boiling constituents are distilled off from the oil phase.

37. The process according to claim 36, which is carried out by means of LDP (liquid dispersion polymer) technology.

38. The process according to claim 35, wherein, during the inverse emulsion polymerization, component b) is added to the oil phase.

39. The process according to claim 35, wherein the temperature during the inverse emulsion polymerization is kept constant or increases.

40. The process according to claim 35, wherein the temperature during the inverse emulsion polymerization is kept constant and is at least 40° C.

41. A surfactant-containing acidic formulation comprising at least one thickener according to claim 21, where the pH of the formulation is 1 to <7.

42. A surfactant-containing acidic formulation according to claim 41 to be used in hair cosmetics, in hairstyling, as a shampoo, as a softener, as a care composition, as a conditioner, as a skin cream, as a shower gel, as a fabric softener for laundry, or as an acidic cleaner.

43. A surfactant-containing alkaline formulation comprising at least one thickener according to claim 21, where the pH of the formulation is 7 to 13.

44. A surfactant-containing alkaline formulation according to claim 43 to be used as a care composition, as a liquid detergent or as a dishwashing detergent for machine washing or hand washing.

45. A thickener according to claim 21 to be used as a viscosity modifier, for optimizing shear dilution, as a thickening agent, for stabilizing suspended ingredients with a size in the range from nanometers to millimeters or in surfactant-containing acidic or alkaline formulations.
